(19) United States
(12) Patent Application Publication
(10) Pub. No.: US 2011/0300553 A1
(43) Pub. Date: Dec. 8, 2011

(54) ENGINEERING COMPLEX MICROBIAL PHENOTYPES WITH SUCCESSIVE INTEGRATIONS OF EXOGENOUS DNA (SIEDNA)

(75) Inventors: Eleftherios T. Papoutsakis, Newark, DE (US); Changhao Bi, Newark, DE (US); Sergios Nicolaou, Newark, DE (US)

(73) Assignee: UNIVERSITY OF DELAWARE, Newark, DE (US)

(21) Appl. No.: 13/151,920

(22) Filed: Jun. 2, 2011

Related U.S. Application Data
(60) Provisional application No. 61/350,690, filed on Jun. 2, 2010.

(51) Int. Cl. C12Q 1/68; C12N 1/21

(52) U.S. Cl. 435/6.15; 435/252.33

(57) ABSTRACT

The present invention relates to a recombinant E. coli exhibiting a complex phenotype, comprising three or more heterologous DNA fragments integrated into a chromosome of the recombinant E. coli bacterium. Also provided are methods for screening such a recombinant E. coli.

### CROSS-REFERENCE TO RELATED APPLICATION

This application claims the benefit of U.S. Provisional Application No. 61/350,690, filed Jun. 2, 2010, the contents of which are incorporated in their entireties.

### REFERENCE TO U.S. GOVERNMENT SUPPORT

This work is supported by a grant from the Office of Naval Research, Department of Defense (Grant No. N000141010161). The United States has certain rights in the invention.

### FIELD OF THE INVENTION

The invention relates generally to engineering complex microbial phenotypes. In particular, the invention relates to recombinant microorganisms exhibiting a desirable complex phenotype as well as methods for engineering these recombinant microorganisms.

### BACKGROUND OF THE INVENTION

Many important properties of a cell to be used for biotechnological applications are the result of a complex integration of metabolic pathways and regulatory/signal transduction events involving many genes, which in most cases are not known. These are referred to as complex microbial phenotypes. There are several important complex phenotypes that one desires to develop for practical applications in the context of metabolic engineering. For example, in bioprocessing and advanced bioremediation applications, in addition to maximizing the flux for a desirable product, the robustness and prolonged productivity of the biocatalyst in the cells, under realistic bioprocessing conditions, are equally important issues. Thus, the ability of cells to withstand “stressful” bioprocessing conditions (some of which have never been encountered by the cells) without loss of productivity, is a very significant goal. Such conditions could include: toxic substrates, accumulation of toxic products & byproducts, high or low pH, and high salt concentrations. Most if not all of these would be encountered in applications for the production of chemicals or biofuels, as well as in bioremediation applications for removal of toxic chemicals from contaminated soil or aqueous systems. The difficulty is that any of these phenotypic traits is determined by several genes or complex regulatory circuits. Complex phenotypes are also encountered when one desires to develop a de novo capability or pathway in a particular cell. For example, a cell may be missing a few enzyme-coding genes and a few regulatory genes necessary to carry out biosynthesis or degradation of a desirable chemical, but it is unknown which genes are missing.

A major concern in biological production of chemicals and fuels (but also in bioremediation applications and whole-cell biocatalysis in various organic media) is the inhibitory effects of toxic products, byproducts or impurities in the substrates used. There has been limited progress in development of alcohol, or other-solvent tolerant strains. The tolerance phenotype is the result of several simultaneous mechanisms of action, including molecular pumps, changes in membrane properties, changes in cell wall composition, altered energy metabolism, changes in cell size and shape, whereby the mode of action may be independent from each other. Tolerance of microorganisms to chemicals is a complex, multigenic, and extremely heterogeneous trait, which is affected by several process parameters such as pH, temperature, osmotic pressure, and the presence of other small molecules. Thus, development of strains with superior tolerance characteristics to specific chemicals and general stressful bioprocess conditions is an important and widely recognized goal not only in the context of production of chemicals and biofuels from carbohydrates, but also for many bioremediation applications.

Lactobacilli include some of the most ethanol-, butanol- and generally alcohol-tolerant known organisms despite the perception that yeasts are overall more tolerant to ethanol. However, the molecular basis of their tolerance remains unknown. To develop new complex phenotypes contributing for tolerance in E. coli, multiple genes from L. plantarum are to be integrated into the E. coli chromosome. However, there has been no convenient method serving this purpose so far. Commonly used methods for DNA integration in E. coli utilize Lambda Red recombinase assisted homologous recombination mechanism. The efficiency of the homologous integration dropped very quickly as the size of the integrated DNA fragment increases, making this method not suitable for an integration library.

The ability to generate cellular phenotypes, which are determined by complex interactions among genes and other genetic and epigenetic elements, is an important goal in modern biology and biotechnology. There remains a need for a suitable method to generate a complex phenotype in a host microorganism where the molecular basis of the phenotype is unknown.

### SUMMARY OF THE INVENTION

The present invention relates to a recombinant E. coli exhibiting a complex phenotype and a method for constructing and screening such a recombinant E. coli.

A recombinant E. coli bacterium exhibiting a desirable complex phenotype is provided. The recombinant E. coli bacterium comprises three or more heterologous DNA fragments integrated into its chromosome.

The heterologous DNA fragments may be derived randomly from one or more heterologous prokaryotes. For example, they are derived randomly from one heterologous prokaryote.

The heterologous prokaryotes are in one or more genera selected from the group consisting of Antinomies, Bacillus, Clostridium, Deinococcus, Escherichia, Klyveromyces, Lactobacillus, Nocardioides, Pichia, Pseudomonas, Rhodococcus, Saccharomyces, Streptomyces, and Sterigmatomyces. Preferably, the heterologous DNA fragments are derived randomly from Lactobacillus plantarum.

The heterologous DNA fragments may be of any size. Preferably, they are 3 kb or larger. The heterologous DNA fragments may be derived from one gene.

The recombinant E. coli bacterium may comprise three or four integrated heterologous DNA fragments.

The complex phenotype may be tolerance to a toxic chemical, metabolite, substrate, high or low pH, oxidative stress, or bioprocess condition. The toxic chemical may be selected from the group consisting of a solvent, a carboxylic acid, a hydrocarbon, a phenolic compound, a halogenated organic chemical, and a toxic salt or metal ion. The toxic chemical is preferably ethanol, butanol or isopropanol.

A method for screening a recombinant E. coli bacterium exhibiting a desirable complex phenotype is also provided. The method comprises continuously integrating a heterologous DNA fragment into a chromosome of a host E. coli bacterium, whereby the host E. coli bacterium comprises three or more integrated heterologous DNA fragments.

In the method of the present invention, the heterologous DNA fragments are derived randomly from one or more heterologous prokaryotes. For example, they may be derived randomly from one heterologous prokaryote. The heterologous DNA fragments may be of any size, preferably 3 kb or larger. They may also be derived from one gene.

In the method of the present invention, the heterologous prokaryotes may be in one or more genera selected from the group consisting of Antinomies, Bacillus, Clostridium, Deinococcus, Escherichia, Klyveromyces, Lactobacillus, Nocardioides, Pichia, Pseudomonas, Rhodococcus, Saccharomyces, Streptomyces, and Sterigmatomyces.

In the method of the present invention, the continuously integrating step may comprise three rounds of integrations, whereby the host E. coli bacterium comprises three integrated heterologous DNA fragments.

In the method of the present invention, the continuously (or successively) integrating step may comprise four rounds of integrations, whereby the host E. coli bacterium comprises four heterologous DNA fragments.

In the method of the present invention, the continuously integrating step may comprise three or more rounds of integrations, whereby three or more heterologous DNA fragments are integrated in the host E. coli bacterium.

### BRIEF DESCRIPTION OF THE DRAWINGS

FIG. 1 illustrates continuous integrations of heterologous DNA fragments into a chromosome of a host microorganism. As shown, four (4) rounds of continuous integrations of L. plantarum DNA fragments are integrated into the chromosome of E. coli.

FIG. 2 illustrates steps to construct the linear DNA for a second step integration of Cm gene along with a first L. plantarum DNA fragment.

FIG. 3 shows colony PCR to confirm that the Cm gene along with a 3 kb L. platarum DNA fragment inserted a Km gene, and to identify the integrated 3 kb L. platarum DNA fragment. The position of the primers used for confirmation was represented by the arrow symbol placed on genetic segments.

FIG. 4 shows two alternate and/or parallel selection protocols for developing desired complex phenotypes.

FIG. 5 shows construction of a first plasmid for a random insertion of foreign DNA and an antibiotic resistance marker into an E. coli chromosome.

FIG. 6 illustrates a consecutive integration process with Cre-loxP RMCE. In the first round integration, the loxPwt and loxPa1 sequences on the plasmid fragment loxPwt-cm-loxPb1-LP1-loxPa2 recombine with their counterparts on loxPwt-sp-loxPa1 in the chromosome. After the recombination, a loxPwt-cm-loxPb1-LP1-loxPa3 sequence was incorporated into the fruk locus and replaced the loxPwt-sp-loxPa1 sequence. With the same mechanism, the second DNA fragment from L. plantarum, LP2, along with antibiotic marker tet and the third DNA fragment from L. plantarum, LP3, along with antibiotic marker gm were integrated in the genomic loci.

FIG. 7 shows PCR confirmation of three rounds of RMCE integrations: (A) cm in E. coli EC100cmLP strains; (B) first L. plantarum DNA fragment in EC100CmLP strains; (C) tet in EC100tetLP2 strains; (D) second L. plantarum DNA fragment in EC100tetLP2 strains; (E) first L. plantarum DNA fragment in EC100tetLP2 strains; and (F) gm in E. coli EC100gmLP3 strains.

FIG. 8 shows PCR amplified L. plantarum DNA in the EC100CmLP libraries enriched with different conditions: (A) three rounds of 35 g/L ethanol; (B) one round of 35 g/L ethanol and one round of 40 g/L ethanol; (C) two rounds of 35 g/L ethanol and one round of 45 g/L ethanol.

### DETAILED DESCRIPTION OF THE INVENTION

The present invention is based on the discovery of methods for continuously integrating multiple heterologous DNA fragments into E. coli such that the engineered E. coli exhibits a desirable complex phenotype. As a result, multiple heterologous DNA fragments are integrated into the chromosome of a host E. coli bacterium.

According to one aspect of the present invention, a recombinant microorganism exhibiting a desirable phenotype is provided. The recombinant microorganism comprises three or more heterologous DNA fragments integrated into one or more of its chromosomes.

The recombinant microorganism may be a bacterium, yeast or more broadly fungus. Examples of the bacteria include Gram negative and Gram positive bacteria, for example, microorganisms of the genera Clostridium, Bacillus, Lactobacillus, Radiococcus, Escherichia, Listeria, and Staphylococcus. Preferably, the microorganism is E. coli. The fungus may be in the genera Saccharomyces, Klyveromyces, Pichia, or Sterigmatomyces.

The recombinant microorganism may comprise three, four, five or more integrated heterologous DNA fragments. The heterologous DNA fragments may be integrated into one, two, three, four, five or more chromosomes of the recombinant microorganism. Preferably, the recombinant microorganism is a recombinant E. coli comprising three, four, five or more heterologous DNA fragments integrated in its chromosome.

The heterologous DNA fragments may be of any size. Preferably, they may be 3 kb or larger.

The heterologous DNA fragments may also be derived from one or more genes. For example, they may be derived from one gene.

The heterologous DNA fragments may be derived randomly from one or more other (or heterologous) organisms. The heterologous organism may be an eukaryote or prokaryote, preferably, a prokaryote. The heterologous organism may exhibit the desirable phenotype.

The one or more heterologous prokaryotes may be in genera Actinomyces, Bacillus, Clostridium, Deinococcus, Escherichia, Klyveromyces, Lactobacillus, Nocardioides, Pichia, Pseudomonas, Rhodococcus, Saccharomyces, Streptomyces, and Sterigmatomyces. For example, the heterologous prokaryote may be Lactobacillus plantarum (L. plantarum) or Lactobacillus bravis, preferably L. plantarum. The heterologous prokaryote may exhibit the desirable phenotype.

The phenotype may be simple or complex. Preferably, the phenotype is complex. A simple phenotype refers to a phenotype modulated by a single functional gene while a complex phenotype refers to a phenotype modulated by two or more (e.g., two, three, four, five or more) functional genes. In general, a complex phenotype may involve multiple cellular structures and mechanisms, for example, membrane integrity, cell wall composition, synthesizing molecular pumps, alternating energy metabolism as well as other unknown mechanisms. Multiple genes and their interactions constitute the genetic basis of complex phenotypes.

Examples of complex phenotypes include tolerance to a toxic chemical, metabolite, substrate, high or low pH, oxidative stress, and/or bioprocess condition. A toxic chemical may be present in feedstock. It may also be a toxic side product, or an end product produced during a bioprocess or fermentation. Examples of toxic chemicals include a solvent, carboxylic acid, hydrocarbon, phenolic compound, halogenated organic chemical, and toxic salt or metal ion. Preferably, the toxic chemical is ethanol, butanol or isopropanol. The toxic chemicals may be useful for industrial applications, biofuel applications, and applications in industrial biocatalysis or bioremediation.

According to another aspect of the present invention, a method for screening a recombinant microorganism exhibiting a desirable phenotype is provided. The method comprises continuously integrating a heterologous DNA fragment into one or more chromosomes of a host microorganism. As a result, the host E. coli bacterium comprises three or more integrated heterologous DNA fragments.

In the methods in accordance with the present invention, the three or more heterologous DNA fragments may be derived randomly from one or more other (or heterogonous) organisms. They may also be of any size. They may further be derived from one or more genes.

The method may comprise three, four, five or more rounds of integrations of a heterologous DNA fragment into one, two, three, four, five or more chromosomes of the host microorganism, whereby the host microorganism comprises three, four, five or more heterologous DNA fragments. Preferably, the method comprises three, four, five or more rounds of integrations of a DNA fragments into the chromosome of a host E. coli bacterium, whereby the host E. coli bacterium comprises three, four, five or more heterologous DNA fragments.

The method may comprise consecutively integrating heterologous DNA fragments into E. coli or other bacterial chromosomes using the Cre LoxP system. This technique includes inducible Cre expression on a helper plasmid, a non-replicable plasmid vector set comprising a special construct with mutated loxP sequences and a workable antibiotic marker, in combination with specific manipulation protocols developed for successful integration and confirmation.

In accordance with the present invention, upon integration of heterologous DNA fragments into a host microorganism (e.g., E. coli), genes in the heterologous DNA fragments are expressed and the host microorganism acquires a desirable phenotype, simple or complex. The genes in the heterologous DNA fragments contributing to the desirable phenotype in the recombinant microorganism may be identified by sequencing the heterologous DNA fragments present in the recombinant microorganism. Such identified genes may be introduced into other microorganisms to improve or introduce the desirable phenotype in the other microorganisms.

The methods in accordance with the present invention may be used to identify and generate recombinant microorganisms (e.g., E. coli) that have novel catabolic capabilities, such as the ability to degrade cellulose or xylan or other complex carbon sources.

The methods in accordance with the present invention may also be used to identify and generate recombinant microorganisms (e.g., E. coli) that have novel anabolic capabilities, such as the ability to produce any desirable chemical or compound, by identifying either previously unknown pathways for the specific chemical or by identifying limiting enzymatic steps for the production of the chemical.

### EXAMPLE 1

### Building Complex Phenotypes by Accelerated Evolution to Generate “New” Genomes

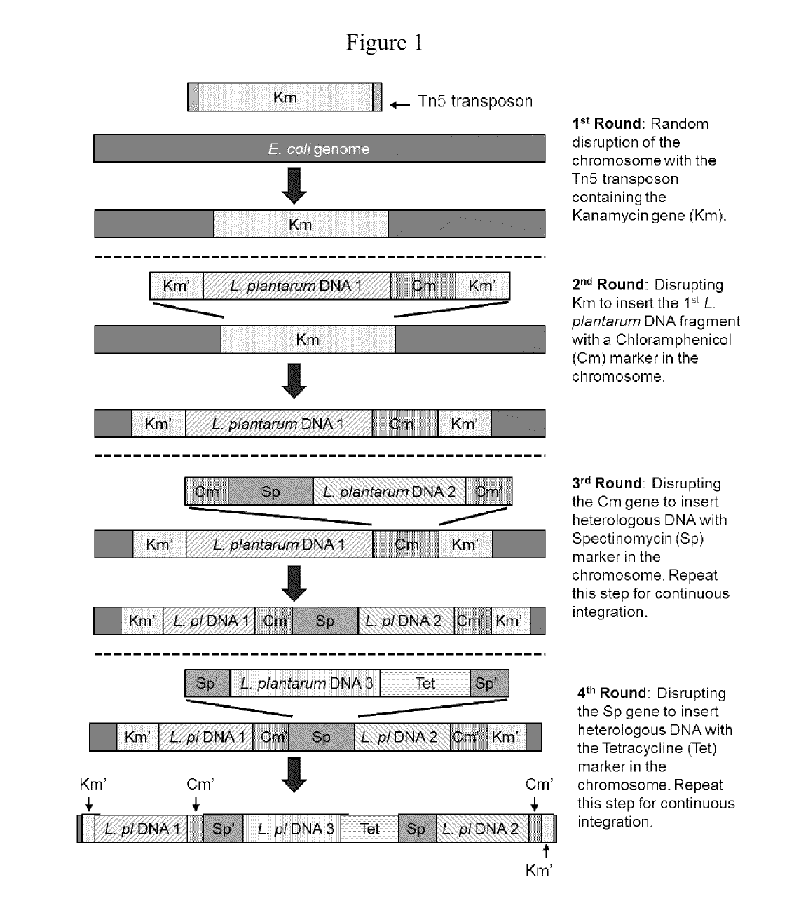

DNA genomic-libraries may be developed for generating random gene knockouts and DNA insertions into the E. coli chromosome as a means to build complex phenotypes. The idea is to use homologous recombination for random integration into and disruption of the E. coli genome by a cassette consisting of an antibiotic resistance marker along with a heterologous (or homologous if so desired) genomic DNA fragment. A DNA library is created using genomic DNA from an organism whose DNA desired for integration into E. coli. After a first round of random DNA integration into the E. coli chromosome, a diverse E. coli population is generated to contain random integrated DNA segments from the heterologous DNA library. Additional DNA can be integrated in subsequent steps. This is achieved by targeting the initial antibiotic resistance marker used in the first round of recombination as an integration site, and using a different marker for selection. For a third round of chromosomal integration, the second antibiotic marker (the first antibiotic marker was already largely deleted from the chromosome) is targeted as a region of homology and another antibiotic marker is integrated along with additional genomic DNA. This process can be repeated until the desired number of chromosomal inserts is achieved in order to enlarge the E. coli genome and increase the diversity of the inserted DNA. Following three or more rounds of DNA integrations, the resulting mutant cells can be screened for a desirable phenotype, specifically, tolerance. The combination of random gene disruptions coupled with multiple rounds of insertion of heterologous DNA will generate a large population of “different genomes,” which can then screened for complex phenotypes. The continuous integration steps are illustrated in FIG. 1.

First Step. Initial chromosomal disruption and insertion of genomic DNA as Round 1. The Tn5 transposon (Epicentre) was used to generate the E. coli K12 and an E. coli Epi300 knock-out library with the kanamycin resistance gene (Km) randomly inserted on the chromosome. The K12 library contains about 2000 individual knock-out strains and the Epi300 library contains about 20000 strains. Out of the K12 library, 6 colonies were randomly picked out to identify the chromosomal insertion sites to demonstrate the diversity of the library. As indicated in Table 1, all 6 strains had the Tn5 cassette inserted at different locations on the chromosome.

TABLE 1
The insertion locations of the Tn5 cassettes on K12 chromosome.

| K12 Tn5 mutants | Tn5 cassette insertion location |
|---|---|
| 1 | 3746904 |
| 2 | 496291 |
| 3 | 4149749 |
| 4 | 3860285 |
| 5 | 3119251 |
| 6 | 4060121 |

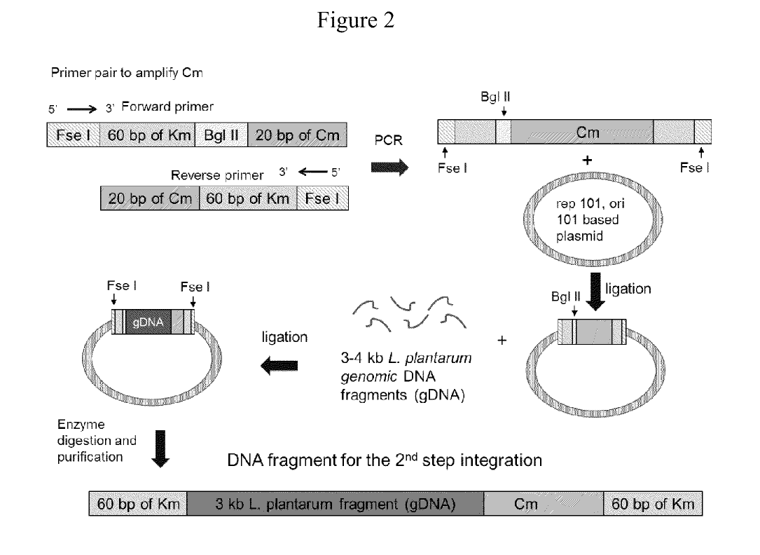

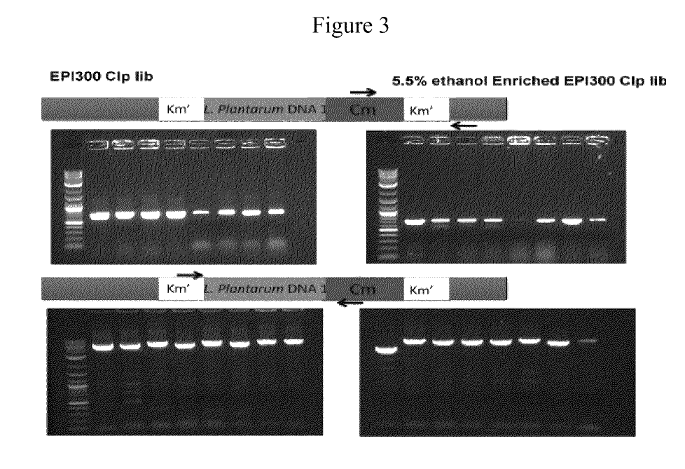

Second Step. Utilizing the first antibiotic resistance marker as a gateway for the insertion of more genomic DNA into the E. coli chromosome as Round 2, 3, etc of DNA chromosomal integration. After the first Round of disruption/integration into the chromosome, the selection marker will serve as the target region of homology so that additional DNA can be inserted into the antibiotic resistance gene. For the second round of integration, a linear DNA fragment containing the Chloramhenicol (Cm) gene along with a random 3 kb fragment of L. plantarum DNA was constructed. This fragment is flanked by 70 base pairs on the 5′ and 3′ ends that are homologous to the Km gene segments, thus targeting the Km gene for homologous recombination and disruption. To construct this linear DNA in large amounts and to prevent plasmid contamination for the transformation, a rep101-ori101 based plasmid was chosen as plasmid backbone. These plasmids are not stable in E. coli at temperature higher than 30° C. As described in FIG. 2, long PCR primers with Km gene sequence and cloning site were designed to clone the Cm gene. The PCR product was ligated to rep101-ori101 based plasmid backbone. The plasmid was digested by Bgl II, and ligated to 3 kb L. plantarum genomic DNA. The constructed plasmid library was then digested by Fse I to obtain the linear DNA for electroporation of the E. coli mutant library constructed in the first round containing the Red Helper plasmid pKD46 (FIG. 1). The same protocol was used to construct linear DNA containing spectinomycin resistant gene or tetracycline gene to integrate into resistance gene present in parent strain library for the third and fourth rounds (FIG. 3). Multiple rounds of insertions can be thus performed.

The second round integration library with Cm gene and the first 3 kb L. plantarum genomic DNA inserted into chromosome of E. coli Epi300 host were successfully established with about 1000 individual strains. The library was cultured at 42° C. for 27 generations to eliminate any plasmid contamination. Although the individual strains constitute the library could not reach 1× coverage of the total L. plantarum genome, this small scale library established here was for proof of the concept only. The coverage of the integration library can be expanded by optimizing the efficiency of experimental process or simply by combining more transformations together.

To confirm the recombination events illustrated in FIG. 1 round 2, where the Cm gene along with a 3 kb L. Platarum DNA fragment is inserted into the Km gene, colony PCR were performed. The results are illustrated in FIG. 3. The amplified integrated DNA fragments were sequenced to be 4 kb L. platarum DNA. Both original library and 5.5% ethanol enriched library were analyzed. The sequencing results of inserted L. plantarum DNA are summarized in Tables 2 & 3.

TABLE 2
The sequences of L. platarum WCFS1 DNA fragments integrated in the
original second round integration library.

| Sample | Start | End | | |
|---|---|---|---|---|
| number | position | position | length | Featured genes |
| 1 | 1899576 | 1894976 | 4600 | Conserved hypothetical |
| 2 | 592144 | 596972 | 4828 | protein prophage Ip1 |
| 3 | 1727178 | 1721789 | 5389 | ABC transporter, clp protease |
| 4 | 1931776 | 1927192 | 4584 | cell division protein ftsw, |
| | | | | pruvate carboxylase |
| 5 | 1727180 | 1721787 | 5393 | ABC transporter, clp protease |

TABLE 3
The sequences of L. platarum WCFS1 DNA fragments integrated in the
5.5% ethanol enriched second round integration library

| Sample | Start | End | | |
|---|---|---|---|---|
| number | position | position | length | Featured genes |
| 1 | 1126197 | 1123026 | 3171 | hypothetical protein, priming |
| | | | | glycosyltransferase |
| 2 | 1727181 | 1721803 | 5378 | ABC transporter clp protease |
| 3 | 592142 | 596970 | 4828 | prophage Ip1 |
| 4 | 592148 | 596970 | 4822 | prophage Ip1 |
| 5 | 592136 | 596970 | 4834 | prophage Ip1 |
| 6 | 3001950 | 2996511 | 5439 | Prophage Ip4 protein 8, |
| | | | | purine-cytosine transporter |

It was determined by the results that the small scale second step integration library was successfully constructed with expected genetic features. Because there was a 27 generation culturing to eliminate plasmid contamination, some of the inserted L. Platarum DNA was preferentially enriched in the library, such as the 5390 bp fragment located on L. Platarum WCFS1 chromosome from 1721803 to 1727181. This bias will be eliminated by using non-replicable plasmids to amplify the linear DNA for integration.

Third Step. Screening of the generated E. coli integrant libraries for a desirable complex phenotype. Multiple rounds of insertion of heterologous DNA will be performed to create a library of E. coli integrants. These cells can be screened after each and every step or after a few steps of DNA integration by exposure to selective medium or differential conditions (exposure to a stress vs. not; here: ethanol, or butanol, or 1,2,4-butanetriol) to select cells that have or progressively develop a desirable phenotype. The assumption is that the gene disruptions and DNA insertions will create complex phenotypes that increase tolerance and allow for preferential growth under the applied stress. Thus, the population will eventually be taken over by cells carrying inserts that provide a selective advantage. Briefly, the collection of cells will be stressed with a stressor such as ethanol until stationary phase, and will then be transferred into fresh media with higher stress concentrations. The repeated stress will enrich for tolerant strains, which can be further characterized at a later stage.

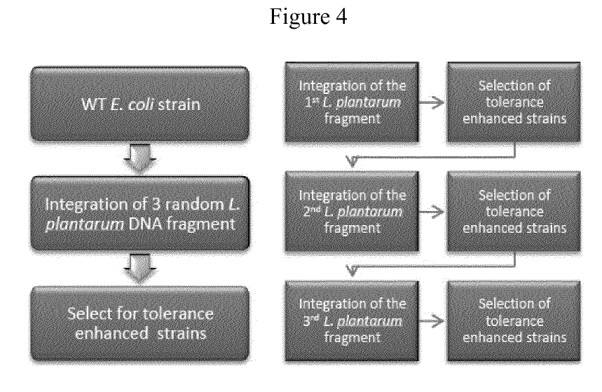

Two alternate or parallel but independent selection protocols were designed for desired phenotypes as illustrated in FIG. 4. In protocol one, the stress selection will be applied after three random heterologous genetic fragments have been integrated into host chromosome. In protocol two, a selection condition is to be applied after each round of genetic integration. The stressful condition enriched library is used as parent strains for next round of genetic integration. For example, the 5.5% ethanol enriched the second round integration library created in step 2 is to be used as parent strain for the third round genetic integration as illustrated in FIG. 4. When the library was cultured with ethanol stress, some of the inserted L. plantarum genetic elements were selectively enriched according to our hypothesis (Table 3). For example, the 4830 bp L. plantarum genetic fragment containing prophage Lp1 was preferentially enriched in the population of the second round integration library after ethanol stress as applied. These enriched L. plantarum genetic elements might be expressed in the E. coli host and contribute to tolerance the ethanol stress.

A small scale second round integration library was constructed. The third round genetic integration library was also constructed and currently under the process of genetic characterization. As expected, the selection protocol was applied and demonstrated that the inserted L. platarum genetic elements contributed to the host tolerant phenotype against ethanol stress. As a result, the detailed protocols for the method had been proved to be feasible.

The repeated genomic integrations allow for the interactions of multiple, distantly located genetic loci to create new complex phenotypes. Different sources of genomic DNA, for example various organisms useful in biotechnology including but not limited to Clostridia, Bacilli, Lactobacilli and Deinococcus radiodurans, can be used to create a hybrid organism that allows for the interactions of genomic elements from many species. Thus, not only are multiple genetic loci are allowed to interact, but those loci can be derived from different organisms as well.

Except for complex phenotypes, desired bio-synthesis or degradation pathways can also be identified using the method we created here. By several rounds of integrations of genetic elements into a host chromosome, there might be one specific strain out of the genetic library of many combinations of genetic elements, which has all the genes in a certain pathway. Then selection condition can be applied to select for such strains. Of course, a very large library must be constructed to contain all the possible combinations of genetic elements in order to contain the right combination of genes of a certain pathway.

In addition to the genomic or subgenomic libraries, one may employ metagenomic libraries, especially libraries that are enriched for a specific desirable phenotype as has been described in the literature. It has been estimated that there are 10³⁰ microbes in the environment and 10³¹ phage particles that can shuttle genomic information between species. This enormous genetic diversity remains to be explored. Microbial communities evolve to survive in their specific environment and can provide the genetic material to identify novel genes with desirable functions. However, the grand majority of bacteria and other microbes cannot be cultured and thus the communities cannot be reproduced in the laboratory. With the method created in this project, linear metagenomic DNA elements can be constructed in order to integrate into the host chromosome. Alternatively, a library of combinations of metagenomic DNA elements can also be established in one plasmid using the method we created. By creation of a library of combinations of metagenomic elements in a host bacterium, desired pathways or complex phenotypes might be able to be identified with appropriate selection conditions.

The recombination integration protocol illustrated in FIG. 1 can be repeated endlessly to create a bigger and more diverse library. This process was similar to genetic evolution. A highly desired phenotype can be enhanced endlessly by continuous integrations of genetic elements into host chromosome.

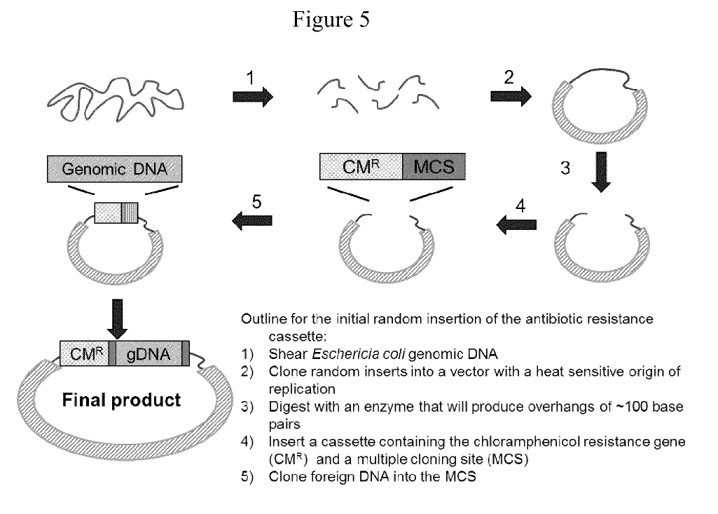

The initial insertion of DNA that can then act as template, or scaffold, for subsequent integration can be achieved by other means, for example using E. coli DNA for the homologous recombination as shown in FIG. 5. To achieve this, genomic DNA from the strain that will be used for the chromosomal insertions can be isolated and sheared into small fragments. Those fragments can then be cloned into a vector containing the chloramphenicol resistance gene and a multiple cloning site (MCS). Additional DNA can then be cloned into the plasmid. The final construct can then be linearized and used for homologous recombination. The overhangs contained in the plasmid will be homologous to the native chromosome, thus allowing for recombination. This method will create random disruptions that may prove beneficial in creating a complex phenotype.

An example would be to utilize a non-antibiotic-resistance gene system to select for integration. Such a system could be based on the galK gene, coding for galactokinase, which is necessary for D-galactose utilization. The galK system allows for both positive and counter selection. During the knockout process, foreign DNA will be inserted in the chromosome to disrupt the galK gene thus achieving the first integration. Counter-selection to remove all cells where the integration did not take place into the galK gene is accomplished by adding in the medium 2-deoxy-galactose (DOG), which is metabolized by the cells that express GalK to generate a toxic product that kills the cells that express GalK. In the second round of integration, the galK gene is reinstated by utilizing a cassette containing the galK gene and additional DNA. The cells with the new round of integration are selected for growth in a medium with galactose. The process can then be repeated. Other systems similar to the galK gene system can be also developed.

One can repeat this where the initial or subsequent steps can target all integrations into specific non-essential genes or synthetic integration sites that we can generate into the chromosome by recombineering. A cassette containing the DNA to be integrated and an antibiotic resistance marker can be flanked with regions of homology targeting a specific non-essential gene. Following homologous recombination, the non-essential gene is disrupted by the additional DNA and the antibiotic resistance marker inserted into the chromosome. The exact location of insertion is known, and this process can be repeated for many non-essential genes. Once the first cassette is inserted (after homologous recombination and the disruption of the non-essential gene(s)), it can then be used as a scaffold for subsequent integrations

At each and every step after integration of heterologous DNA, one can also insert one or two compatible plasmid- or fosmid-based libraries of DNA from metagenomic sources or from other heterologous genomes and screen for the desirable traits. The identified DNA elements on the plasmids or fosmids that help develop the desirable trait can then be integrated selectively into the chromosome.

In the protocol of constructing linear DNA to transform into the target host for recombinational integration as illustrated in FIG. 1, where the plasmid backbone is replaced with an R6k based plasmid. This is a plasmid that can only replicate with the pir gene encoded Pi protein present into the parent strain. Thus, the use of a R6k plasmid (or a similar plasmid that can only replicate under specific conditions) ensures that any undigested plasmid will not be transformed into the host, and increases the probability of successful homologous recombination into the chromosome.

### EXAMPLE 2

### Continuous Integrations of DNA Fragments Using the Cre Lox System

### Materials and Methods

Bacterial growth and maintenance conditions. E. coli strains were grown aerobically at 37° C. or 30° C. in liquid LB media or on solid LB agar plates supplemented with the appropriate antibiotic (ampicillin 50 μg/ml, chloramphenicol 35 μg/ml or 17 μg/ml, spectinomycin 100 μg/ml, or tetracycline 10 μg/ml or 7 μg/ml). E. coli strains were stored at −85° C. in LB supplemented with 15% glycerol.

Analytical methods. Cell growth was monitored by measuring the A₆₀₀ using a DU 730 spectrophotometer (Beckman-Coulter, Brea, Calif.). DNA concentrations and purities were measured at 260 nm and 280 nm using a NanoDrop spectrophotometer (Wilmington, Del.). For DNA with lower concentration, Quant it kit (Invitrogen, Carlsbad, Calif.) was used for measurement.

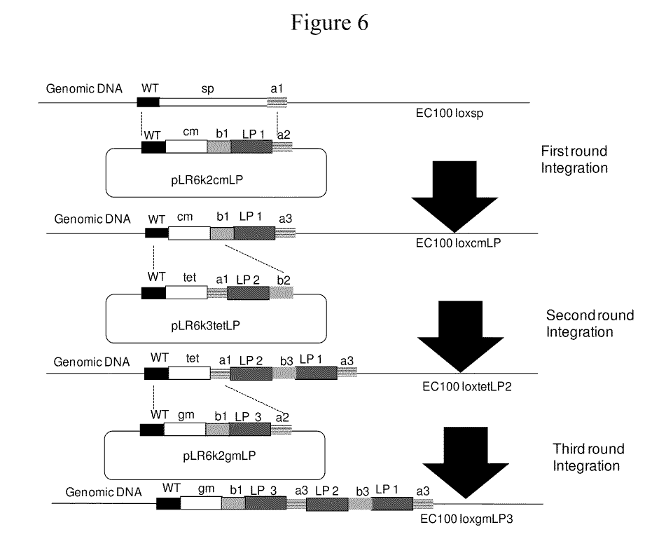

Plasmid construction. PCR was used to amplify the DNA loxPwt-Cm-loxPb1-IRES-loxPa2 fragment out from p2 to ligate to the conditional replicon r6kori from Tn5 cassette (Epicentre, Madison, Wis.), which forms a conditional replicating plasmid pR6k2cm (Table 4). Using the pR6kp2cm as template, PCR was used to amplify the backbone of this plasmid. In addition, a constitutive promoter bla (need a ref and a sort explanation about this promoter) facing outward was added to both ends of the PCR product. L. plantarum DNA fragments of 2 to 5 kb were ligated to the PCR product to form the plasmid library pLR6k2cmLP. Plasmid libraries pLR6k3tetLP and pLR6k2gmLP were constructed using the same approach (FIG. 6).

TABLE 4
Strains and plasmids used

| Strains and | | Source and |
|---|---|---|
| plasmids | Description | Reference |
| E. coli | F-mcrA Δ(mrr-hsdRMS-mcrBC) | Epicentre |
| EC100 | Φ80dlacZΔM15 ΔlacX74 recA1 endA1 | |
| | araD139 Δ(ara, leu)7697 galU galK λ- | |
| | rpsL (StrR) nupG | |
| E. coli | E. coli EC100 fruK:: loxPWT-sp- | Constructed |
| EC100 loxsp | loxPa1 | herein |
| E. coli | E. coli EC100 fruK:: loxPWT-cm- | Constructed |
| EC100cmLP | loxPb1-LP1-loxPa3 | herein |
| E. coli | E. coli EC100 fruK:: loxP-tet-loxPa1- | Constructed |
| EC100tetLP2 | LP2-loxPb3-LP1-loxPa3 | herein |
| E. coli | E. coli EC100 fruK:: loxP-gm-loxPb1- | |
| EC100gmLP3 | LP3-loxPa3-LP2-loxPb3-LP1-loxPa3 | |
| p2 | Plasmid carrying loxPwt-cm-loxPb1- | (Kameyama, |
| | IRES/EGFP-loxPa2 | Kawabe et al. |
| | | 2010) |
| p3 | Plasmid carrying loxPwt-km-loxPa1- | (Kameyama, |
| | IRES/DSRED-loxb2 | Kawabe et al. |
| | | 2010) |
| pJW168 | Plasmid carrying IPTG inducible cre | Lucigen |
| | gene and p101 origin | |
| pR6k2cm | Plasmid carrying loxPwt-cm-loxPb1- | Constructed |
| | IRES/EGFP-loxPa2 cassette and non- | herein |
| | replicable r6k origin | |
| pR6k3tet | Plasmid carrying loxPwt-tet-loxPa1- | Constructed |
| | IRES/DSRED-loxPb2 cassette and non- | herein |
| | replicable r6k origin | |
| pR6k2gm | Plasmid carrying loxPwt-gm-loxPb1- | Constructed |
| | IRES/EGFP-loxPa2 cassette and non- | herein |
| | replicable r6k origin | |
| pLR6k2cmLP | Plasmid library carrying loxPwt-cm- | Constructed |
| | loxPb1-LP-loxPa2 cassette and non- | herein |
| | replicable r6k origin | |
| pLR6k3tetLP | Plasmid library carrying loxPwt-tet- | Constructed |
| | loxPa1-LP-loxPb2 cassette and non- | herein |
| | replicable r6k origin | |
| pLR6k2gmLP | Plasmid library carrying loxPwt-gm- | Constructed |
| | loxPb1-LP-loxPa2 cassette and non- | herein |
| | replicable r6k origin | |

Sequencing primers were designed to sequence the constructed plasmids, pR6k2Cm, and pLR6k2cmLP to confirm the loxP sequences were not mutated during the cloning process. Strains and plasmids are summarized in Table 4.

Genomic Integration and confirmation. To prepare the starting host strain E. coli EC100 loxsp (FIG. 6), the PCR product, fruk′-loxP-sp-loxP1-fruk″ linear DNA fragment, was electroporated into E. coli EC100 (Epicentre, Madison, Wis.) carrying plasmid pKD46 using the Gene Pulser Xcell electroporation system (Bio-Rad, Hercules, Calif.). The primer pair designed as (48 bp fruk′)-(34 bp loxPwt)-(18 bp spF) and (48 bp fruk″)-(34 bp loxPa1)-(18 bp spR) was used to amplify the sp gene out of pCRtopo8 plasmid to obtain the fruk′-loxP-sp-loxP1-fruk″ DNA fragment. Specotinomycin resistant strains were selected and confirmed by PCR reactions using a forward primer in the fruk gene and a reverse primer in the sp gene, as well as a forward primer in the sp gene and a reverse primer in the fruk gene. The PCR products were sequenced to confirm that the loxPwt and loxP1 sequences were intact.

For the first round of integration into the EC100 loxsp strain, the cm gene was used with the first L. platarum DNA fragment. First, the Cre expression plasmid pJW168 (Lucigen, Middleton, Wis.) was transformed into EC100 loxsp. 2 ml of overnight culture with 100 μg/ml Amp was inoculated into 200 ml LB medium with the same Amp concentration. After about 3 hours of culturing at 30° C. (OD₆₀₀ of about 0.05), IPTG was added to the culture to a final concentration of 1 mM. The culture was harvested at OD 0.4. After being washed twice with 150 ml cold DD water and once with 40 ml 10% cold glycerol, the cells were suspended in 500 μl 10% cold glycerol. The prepared electrocompetent cells was electroporated with 2 μg pLR6k2CmLP-library plasmid. After electroporation, the cells were incubated in SOC medium (New England Biolabs, Ipswich, Mass.) with 1 mM IPTG for about 3 hours before plating. Plating was on Cm plates with 1 mM IPTG and 10 mg/ml Cm. The incubation temperature was at 30° C. Some of the colonies on Cm plates were individually plated onto Sp plates to test sensitivity to Sp. The same protocol was used for the second and third round of RMCE integration with tetracycline (Tet) and gentamicin (Gm) as antibiotic markers, respectively.

Colony PCR or PCR using genomic DNA as template was used to confirm the integration and amplify the regions with integrated L. platarum DNA fragments.

Screening of the generated E. coli integrant libraries for enhanced ethanol tolerance and identification of the integrated L. plantarum DNA fragments. The E. coli integrant libraries were screened for survival at increasing concentrations of ethanol. Cells were stressed with an ethanol concentration (starting at 30 g/L) until stationary phase, and were then transferred into fresh media with higher ethanol concentration at small increments. Exposure to and survival at progressively higher ethanol concentrations enriched for ethanol-tolerant strains. Tolerant strains were further characterized using MIC (Minimum Inhibitory Concentration) assays. The integrated L. plantarum DNA fragments in the more tolerant strains were amplified with PCR, and cloned into Topo 2 plasmid for sequencing (Invitrogen, Carlsbad, Calif.).

MIC assays to characterize ethanol-tolerant strains. The library of EC100 loxcmLP in frozen stock with 15% glycerol was first inoculated in LB medium with 30 g/L ethanol plus 8 μg/ml Cm for recovering bacterium activity at 37° C. for overnight culturing. The bacteria were then inoculated into LB medium with a 2% ratio with 35 g/L, 40 g/L, 45 g/L ethanol and cultured for 48 hours. OD₆₀₀ was measured at beginning and end of culturing to see whether the bacteria actually grew or not for determination of MIC. Cultures with growth after 48 hours under ethanol stress were plated to obtain individual colonies, which were subjected for PCR to amplify the integrated L. plantarum DNA fragments.

### Results

Development of the Cre lox system for repeated, consecutive integrations of DNA fragments. The Cre-loxP system is widely used for genetic manipulation in animal cells, but not as frequently in prokaryotes such as in E. coli. Cre recombinase recognizes a specific 34 bp target sequence termed loxP composed of an 8 bp spacer region flanked by two identical 13 bp inverted repeats termed as arm regions. The specificity of RMCE (recombinase-mediated cassette exchange) can be controlled by loxP sequences mutated in the spacer region as shown in Table 5.

TABLE 5
Sequences of loxPs used in the RMCE system.

| | | Spacer | |
|---|---|---|---|
| | Left-arm region | region | Right-arm region |
| loxP[wt] | ATAACTTCGTATA | GCATACAT | TATACGAAGTTAT |
| | (SEQ ID NO: 1) | | (SEQ ID NO: 2) |
| loxPa1 | ATAACTTCGTATA | GTATAGTA | TATACGAACGGTA |
| | (SEQ ID NO: 1) | | (SEQ ID NO: 3) |
| loxPa2 | TACCGTTCGTATA | GTATAGTA | TATACGAAGTTAT |
| | (SEQ ID NO: 4) | | (SEQ ID NO: 2) |
| loxPa3 | TACCGTTCGTATA | GTATAGTA | TATACGAACGGTA |
| | (SEQ ID NO: 4) | | (SEQ ID NO: 3) |
| loxPb1 | ATAACTTCGTATA | GGCTATAG | TATACGAACGGTA |
| | (SEQ ID NO: 1) | | (SEQ ID NO: 3) |
| loxPb2 | TACCGTTCGTATA | GGCTATAG | TATACGAAGTTAT |
| | (SEQ ID NO: 4) | | (SEQ ID NO: 2) |
| loxPb3 | TACCGTTCGTATA | GGCTATAG | TATACGAACGGTA |
| | (SEQ ID NO: 4) | | (SEQ ID NO: 3) |

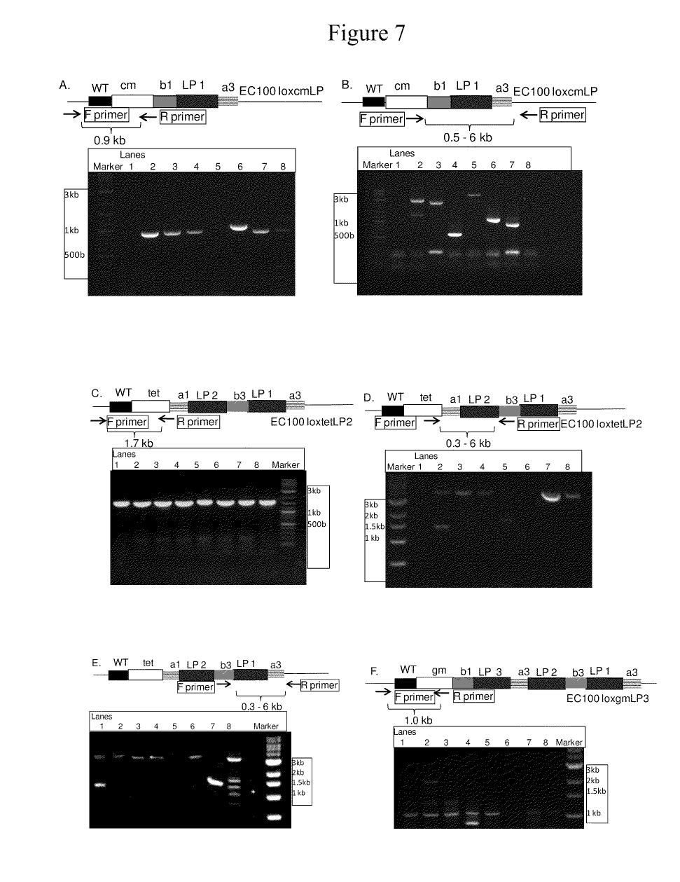

loxPa1 can only recombine with loxPa2 due to the same mutated spacer region. The reaction equilibrium of a RMCE reaction can be controlled by loxPs mutated in the arm regions as indicated in FIG. 7. When mediated by the Cre recombinase, loxPa1 and loxPa2 with 1 mutated arm recombine with each other and generate inactive loxPa3 with 2 mutated arms. Thus, the equilibration of the recombination is pushed towards the loxPa3 end. Similarly, loxPb1 can also only recombine with loxb2 to generate loxb3.

The process of consecutive DNA integrations using RMCE is illustrated in FIG. 6. The loxPWT-sp-loxPa1 DNA fragment was integrated into E. coli EC100 (Table 4) fruk gene with homologous recombination. After the non-replicative plasmid library pLR6k2cmLP was transformed into E. coli EC100 loxsp, facilitated by the Cre expressed from pJW168, the loxPwt and loxPa1 sequences on the plasmid fragment loxPwt-cm-loxPb1-LP1-loxPa2 recombine with their counterparts on loxPwt-sp-loxPa1 in the chromosome. After the recombination, a loxPwt-cm-loxPb1-LP1-loxPa3 sequence was incorporated into the fruk locus and replaced the loxPwt-sp-loxPa1 sequence. The loxPa3 has two mutated arm sequences and no activity with the Cre recombinase. Using the same strategy, the second DNA fragment from L. plantarum, LP2, with antibiotic marker tet can be integrated to replace the cm gene. This process can be repeated to integrate additional DNA fragments in a consecutive fashion.

Chromosomal integration of DNA fragments using the Cre-loxp RMCE. For the first round of DNA-fragment integration, the E. coli EC100 loxsp strain carrying pJW168 was electrotransformed with 1 μg of library pLR6k2cmLP plasmid DNA. After incubation at 30° C. for 48 hours on 10 μg/ml Cm plates, colonies of different sizes appeared as a result of one electrotransformation with 1 μg pLR6k2cmLP DNA were estimated to be 0.5×10⁵ to 10⁵. Since the integrated DNA fragment from L. plantarum has an average size of 3 kb, 10⁵ colonies provided a 100× coverage of the L. plantarum genome. 100 large colonies were randomly picked to test sensitivity to spectinomycin. Out of these 100 colonies, three colonies were found to be resistant to spectinomycin. This suggests that 97 colonies out of 100 colonies on 10 μg/ml-Cm plates lost their spectinomycin resistance, and that these colonies were successful recombinants. The 3 colonies that were resistant to both antibiotics were possibly due to a single crossover integration event at either of the loxP loci, which caused rolling in a whole pLR6k2cmLP plasmid. These single-crossover integration recombinants would keep the original spectinomycin resistance gene and at the same time acquire the tetracycline-resistance gene from the plasmid.

To further confirm the RMCE recombination event and analyze the integrated L. platarum DNA fragments, PCR with a forward primer on the fruk gene and a reverse primer on the tet gene were used to analyze 8 randomly selected colonies. 950 bp PCR products (FIG. 7A) from 6 colonies demonstrated the insertion of the tet gene at the desired chromosomal locus. To amplify the integrated L. platarum DNA, PCR primer pairs at the indicated locations (FIG. 7B) was used to amplify the target region. As shown (FIG. 7B), out of the 8 randomly chosen colonies, 6 had the insertion of a DNA fragment ranging from 500 bp to 6 kb in size. Some of these PCR products were sequenced and confirmed to be L. platarum genomic DNA (data no shown).

The EC100cmLP1 integration library developed above was used directly as a host for the second round of RMCE integration with plasmid library pLR6k3tetLP. After transformation with 1 μg of library plasmid DNA, 10⁵ to 2×10⁵ individual colonies were obtained. The transformation efficiency is similar to the first round integration. Out of 100 tetracycline-resistant colonies examined, 7 were found to be resistant to chloramphenicol, possibly due to a single-crossover integration of pLR6k3tetLP plasmids. PCR was used to confirm the integration of the tet gene into the desired locus. PCR products at the expected size (1.7 kb) were obtained from all 8 random selected colonies, which confirms the successful RMCE recombination (FIG. 7C). The 2^{nd} L. platarum DNA fragment along with the 1^{st} L. platarum DNA fragment was also confirmed to be integrated in the specific loci of the EC100tetLP2 strains (FIG. 7D).

With the same protocol, the EC100tetmLP2 integration library developed above was used directly as a host for the third round RMCE integration with plasmid library pLR6k2gmLP. With 10 μg/ml Gm as selection antibiotic, the integration efficiency was around 2×10⁴ colonies per μg DNA of the pLR6k2gmLP plasmid. Out of 100 colonies, only 1 was found to be resistant to the previous antibiotic marker Tet. PCR products at the expected size (1.0 kb) were obtained from all 8 random selected colonies, which confirms the successful RMCE recombination (FIG. 7F).

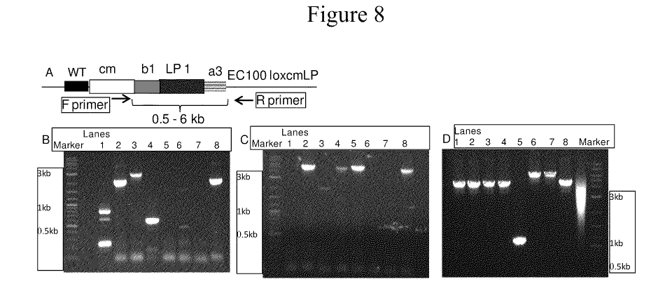

Ethanol pressure selection for tolerance enhanced genes integrated into the E. coli chromosome. To test whether and how the integrated library reacts with ethanol concentration in the medium, the first round integration library, EC100cmLP1, was enriched by culturing with ethanol at concentration of 35 g/L, 40 g/L and 45 g/L. The enriched cultures were plated on Cm plates to obtain single colonies, which were used for colony PCR to determine the integrated L. plantarum DNA in the enriched EC100cmLP1 library. After three rounds of enrichment with medium containing 35 g/L ethanol, integrated L. plantarum DNA at different sizes was still in the enriched library, similar to the un-enriched EC100cmLP1 library (FIG. 9A). After one round of culturing with medium containing 35 g/L ethanol and one round of 40 g/L ethanol enrichment, out of 8 tested colonies, 4 had large L. plantarum DNA insertion raging 4 kb to 6 kb (FIG. 9B). E. coli containing L. plantarum DNA insertion smaller than 2 kb was eliminated in this enriched library. After two rounds of 35 g/L ethanol and 1 round of 45 g/L enrichment, most survived E. coli cells seemed to have large L. plantarum DNA insertion only at two sizes (FIG. 8C). The 4 kb fragment was sequenced to cover the L. plantarum genome from around 1322000 to 1326000, containing genes encoding UDP-N-acetylmuramate-L-alanine ligase, ferrous iron transport protein B, and an ABC transporter (ATP-binding protein). The results suggested ethanol pressure with minimal rounds of transfer tends to enrich for large L. plantarum DNA insertions. Large DNA insertions mean intact genes were able to be expressed in these recombinants and increased the host solvent tolerance.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A recombinant E. coli bacterium exhibiting a desirable complex phenotype, comprising three or more heterologous DNA fragments integrated into a chromosome of the recombinant E. coli bacterium.

2. The recombinant E. coli bacterium of claim 1, wherein the three or more heterologous DNA fragments are derived randomly from one or more heterologous prokaryotes.

3. The recombinant E. coli bacterium of claim 1, wherein the three or more heterologous DNA fragments are derived randomly from one heterologous prokaryote.

4. The recombinant E. coli bacterium of claim 2, wherein the one or more heterologous prokaryotes are in one or more genera selected from the group consisting of Antinomies, Bacillus, Clostridium, Deinococcus, Escherichia, Klyveromyces, Lactobacillus, Nocardioides, Pichia, Pseudomonas, Rhodococcus, Saccharomyces, Streptomyces, and Sterigmatomyces.

5. The recombinant E. coli bacterium of claim 1, wherein the three or more heterologous DNA fragments are derived randomly from Lactobacillus plantarum.

6. The recombinant E. coli bacterium of claim 1, wherein the three or more heterologous DNA fragments are 3 kb or larger.

7. The recombinant E. coli bacterium of claim 1, wherein the three or more heterologous DNA fragments are derived from one gene.

8. The recombinant E. coli bacterium of claim 1, comprising three integrated heterologous DNA fragments.

9. The recombinant E. coli bacterium of claim 1, comprising four integrated heterologous DNA fragments.

10. The recombinant E. coli bacterium of claim 1, wherein the complex phenotype is tolerance to a toxic chemical, metabolite, substrate, high or low pH, oxidative stress, or bioprocess condition.

11. The recombinant E. coli bacterium of claim 10, wherein the toxic chemical is selected from the group consisting of a solvent, a carboxylic acid, a hydrocarbon, a phenolic compound, a halogenated organic chemical, and a toxic salt or metal ion.

12. The recombinant E. coli bacterium of claim 10, wherein the toxic chemical is ethanol, butanol or isopropanol.

13. A method for screening a recombinant E. coli bacterium exhibiting a desirable complex phenotype, comprising continuously integrating a heterologous DNA fragment into a chromosome of a host E. coli bacterium, whereby the host E. coli bacterium comprises three or more integrated heterologous DNA fragments.

14. The method of claim 13, wherein the three or more heterologous DNA fragments are derived randomly from one or more heterologous prokaryotes.

15. The method of claim 13, wherein the three or more DNA fragments are derived randomly from one heterologous prokaryote.

16. The method of claim 13, wherein the three or more heterologous DNA fragments are 3 kb or larger.

17. The method of claim 13, wherein the three or more heterologous DNA fragments are derived from one gene.

18. The method of claim 14, wherein the one or more heterologous prokaryotes are in one or more genera selected from the group consisting of Actinomyces, Bacillus, Clostridium, Deinococcus, Escherichia, Klyveromyces, Lactobacillus, Nocardioides, Pichia, Pseudomonas, Rhodococcus, Saccharomyces, Streptomyces, and Sterigmatomyces.

19. The method of claim 13, wherein the continuously integrating step comprises three rounds of integrations, whereby the host E. coli bacterium comprises three integrated heterologous DNA fragments.

20. The method of claim 13, wherein the continuously integrating step comprises four rounds of integrations, whereby the host E. coli bacterium comprises four integrated heterologous DNA fragments.


SEQUENCE LISTING (4 sequences)

SEQ ID NO: 1 - DNA, 13 bp, Artificial Sequence
  Synthetic sequence
ataacttcgtata

SEQ ID NO: 2 - DNA, 13 bp, Artificial Sequence
  Synthetic sequence
tatacgaagttat

SEQ ID NO: 3 - DNA, 13 bp, Artificial Sequence
  Synthetic sequence
tatacgaacggta

SEQ ID NO: 4 - DNA, 13 bp, Artificial Sequence
  Synthetic sequence
taccgttcgtata